(19) United States
(12) Patent Application Publication
(10) Pub. No.: US 2024/0206928 A1
(43) Pub. Date: Jun. 27, 2024

(54) MINIMALLY INVASIVE RIB FRACTURE REPAIR

(71) Applicant: Adam J. Hansen, Bridgeport, WV (US)

(72) Inventor: Adam J. Hansen, Bridgeport, WV (US)

(21) Appl. No.: 18/544,602

(22) Filed: Dec. 19, 2023

Related U.S. Application Data
(60) Provisional application No. 63/434,567, filed on Dec. 22, 2022.

(51) Int. Cl. A61B 17/80; A61B 17/86; A61B 17/88

(52) U.S. Cl. CPC A61B 17/8076; A61B 17/8004; A61B 17/8023; A61B 17/808; A61B 17/8085; A61B 17/86; A61B 17/8875; A61B 17/8897; A61B 2017/00238

(57) ABSTRACT

Embodiments of an adjustable rib plate apparatus and method are disclosed for rib fracture fixation and repair. The apparatus has a center plate and first and second outer plates that are each attached to but movable relative to the center plate. A pair of bone screws with detachable guidance cables are installed on each of two bone fragments on separate sides of a fracture. The guidance cables are used to guide the first and second outer plates to respective destinations. Locking screws are also guided using the guidance cables and installed in the heads of the bone screws to secure the outer plates. Removable alignment cables with slip knot loops are used to align the center plate with the first and second plates as well as align the bone fragments associated with the fracture. Once aligned, additional bone screws are installed in the first and second outer plates to fully fixate the fracture.

### CROSS-REFERENCE TO RELATED PATENT APPLICATION

This application claims the benefit of and priority to U.S. Provisional Application No. 63/434,567, filed on Dec. 22, 2022, which is incorporated herein by reference.

### FIELD OF THE INVENTION

The present disclosure generally relates to thoracic orthopedic surgery, and more particularly, to apparatus and methods for performing a minimally invasive rib fracture repair using fixation with an adjustable rib plate apparatus.

### BACKGROUND OF THE INVENTION

FIGS. 1-3 illustrate a typical rib fracture associated with a ribcage 9. When a rib fracture 11 occurs as shown in FIG. 1, muscle spasms occur due to the pain and the actual blunt force trauma. What this creates is a displaced rib fracture 11′ or 11″, as shown in FIGS. 2 and 3, respectively. The bony segments are not in line with each other any longer. They can be badly displaced as shown in FIG. 2 or be badly telescoped upon one another as shown in FIG. 3. Nondisplaced rib fractures are common, less damaging, and usually treated nonoperatively, whereas displaced rib fractures are more clinically significant. Displaced fractures can lead to a significant amount of damage internally as well, most commonly lung lacerations, torn intercostal blood vessels, and sheared off intercostal nerves.

### SUMMARY OF THE INVENTION

Various embodiments are disclosed of apparatus and methods for performing a minimally invasive rib fracture repair using fixation with an adjustable rib plate apparatus.

One embodiment, among others, is method for rib fracture fixation and repair. The method uses an adjustable rib plate apparatus having a center plate and first and second outer plates that are each attached to but movable relative to the center plate. A pair of bone screws with detachable guidance cables are installed on each of two bone fragments on separate sides of a fracture. The guidance cables are used to guide the first and second outer plates to respective destinations. Locking screws are also guided using the guidance cables and installed in the heads of the bone screws to secure the outer plates. Alignment cables with slip knot loops are used to align the center plate with the first and second plates as well as align the bone fragments associated with the fracture. Once aligned, additional bone screws are installed in the first and second outer plates to fully fixate the fracture.

Another embodiment, among others, is an adjustable rib plate apparatus. The adjustable rib plate apparatus has a center plate and first and second outer plates. The enter plate has an elongated body with opposing sides that are generally planar. The opposing sides have a periphery defined by opposing side edges and opposing ends. The first outer plate has an elongated body with opposing sides that are generally planar. The opposing sides have a periphery defined by opposing side edges and opposing ends. The second outer plate has an elongated body with opposing sides that are generally planar. The opposing sides having a periphery defined by opposing side edges and opposing ends.

The apparatus further includes a first and second attachment means. The first attachment means (a) attaches the first outer plate to the center plate at a location near respective ends of the first outer plate and the center plate and (b) permits the first outer plate to be movable in a geometric plane that is generally parallel to the geometric planes associated with the opposing sides of the center plate. The second attachment means (a) attaches the second outer plate to the center plate at a location near respective ends of the second outer plate and the center plate and (b) permits the second outer plate to be movable in a geometric plane that is generally parallel to the geometric planes associated with the opposing sides of the center plate.

Another embodiment, among others, is a guidance screw assembly. The assembly includes first and second screws and a segment of flexible cable. The first screw has an elongated body with turning head and tip at opposing ends. The first screw body has outer threads. The first screw ha an inner cannula with inner threads. The second screw has an elongated body with a turning head and tip at opposing ends. The second screw body has outer threads that match the inner threads so the second screw can be driven into the cannula. The segment of flexible cable extends through the elongated body of the second screw between the turning head and the tip, and further extends to and is detachably secured to the first screw. Although not limited to this application, the guidance screw assembly is useful in guiding, placing, and installing the adjustable rib plate apparatus over a rib fracture.

Another embodiment, among others, is a method, or medical procedure, for repairing a rib fracture using the adjustable rib plate apparatus and first and second pairs of guidance screws. The method can be summarized as follows: (a) attaching first and second alignment cables having alignment cable loops with slip knots to the adjustable rib plate apparatus, the first alignment cable loop passing through the first sliding hole of the center plate and an outmost screw hole of the first outer plate, the second cable loop passing through the first sliding hole of the center plate and an outmost screw hole of the second outer plate; (b) installing each of the bone screws of the first and second pairs of guidance screws into first and second rib parts, respectively; (c) passing each of the guidance cables from the first and second pairs of guidance screws, respectively, through first and second screw holes of the first and second outer plates, respectively, and guiding the adjustable rib apparatus against the first and second rib parts; (d) guiding each of the locking screws with respective guidance cables to respective bone screws and securing the first and second outer plates of the adjustable rib plate apparatus to the first and second rib parts, respectively, by installing the locking screws into the bone screws; (e) detaching the guidance cables from the bone screw and the locking screw and removing the guidance cables; (f) tightening the slip knot alignment cable loops in order to bring the first and second rib parts in closer alignment by pulling on the alignment cables; (g) installing a plurality of additional bone screws through respective screw holes in the first and second outer plates and into the first and second rib parts, respectively; and (h) detaching the alignment cables from the adjustable rib plate apparatus and removing the alignment cables.

Another embodiment, among others, is an extended steerable screwdriver. The extended steerable screwdriver has an elongated body extending between first and second ends. A rotatable handle is situated at the first end of the elongated body. A drive shaft passes through the elongated body and is rotated by the handle. A flexible segment associated with the drive shaft enables the drive shaft to bend. There is a worm gear that is driven by the drive shaft. An offset driver having a removable cannulated tip is driven by the worm gear. The tip is designed to engage and drive a turning head of a guidance screw while enabling a tether cable associated with the guidance screw to extend from the turning head of the guidance screw into, through, and out of the driving tip. Finally, an elongated sway bar extends along the elongated body. The sway bar has a universal joint with thumb screw knob designed so that when the thumb screw knob is turned, the sway bar is inwardly or outwardly moved so that the sway bar angularly bends the driver at the flexible segment so that the driver can be guided and steered angularly. A similar mechanism that accomplishes the same controls could be miniaturized to fit all within a single elongated body.

Another embodiment, among others, is a plate positioner and stabilizer. The plate positioner/stabilizer has an elongated body extending between a proximate end and a working distal end. First and second elongated shafts extend along the body. A handle is equipped with first and second control mechanisms that are designed to respectively and independently move the first and second shafts in a longitudinal direction along the body. A positioner head that is attached to and is pivoted by the first and second elongated shafts when moved in the longitudinal direction. The positioner head has a plurality of screws for detachably mounting to the rib plate.

Other embodiments, apparatus, systems, methods, features, and advantages of the present invention will be apparent to one with skill in the art upon examination of the following drawings and detailed description. It is intended that all such additional embodiments, apparatus, systems, methods, features, and advantages be included within this disclosure, be within the scope of the present invention, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a view of a typical rib fracture.

FIG. 2 is a view of the rib fracture of FIG. 1 that has been displaced due to muscle spasms.

FIG. 3 is a view of the rib fracture of FIG. 1 that has telescoped due to muscle spasms.

FIG. 4A is a first top view of an adjustable rib plate apparatus in accordance with the present disclosure.

FIG. 4B is a second top view of the adjustable rib plate apparatus of FIG. 4A.

FIG. 5A is a first side view of the adjustable rib plate apparatus of FIG. 4A, showing a first length and curvature suitable for placement on a concave internal surface of a rib.

FIG. 5B is a second side view of the adjustable rib plate apparatus of FIG. 4A, showing a second length and curvature suitable for placement on a convex external surface of a rib.

FIG. 6 is a view of the guidance screw assembly of the present disclosure with a locking screw tethered by a guidance cable and unengaged with a bone screw, which assembly assists in guiding placement of the adjustable rib plate apparatus of FIG. 4A onto a rib fragment.

FIG. 7 is a view of the guidance screw assembly of the present disclosure with the locking screw engaged with the bone screw, which assembly assists in guiding placement of the adjustable rib plate apparatus of FIG. 4A onto a rib fragment.

FIG. 8 is a view of an extended steerable screwdriver of the present disclosure, which is useful and steering the guidance screw assembly of FIG. 6.

FIG. 9 is a view of the extended steerable screwdriver of FIG. 8 showing the guidance cable of FIG. 6 extending through a cannulated tip of the screwdriver.

FIG. 10 is a view showing installation of two of the four bone screws with respective guidance cables by using the extended steerable screwdriver of FIG. 8.

FIG. 11 is a view showing installation of the fourth bone screws with respective guidance cables by using the extended steerable screwdriver of FIG. 8.

FIG. 12 is a view showing installation of the four bone screws with respective guidance cable upon removal of the extended steerable screwdriver of FIG. 8.

FIG. 13 is a view showing guidance of the adjustable rib plate apparatus of FIG. 4A with the guidance cables to a position across the fracture.

FIG. 14 is a view of the adjustable rib plate apparatus of FIG. 4A that is fully installed on rib fragments to secure and repair the rib fracture.

FIGS. 15 through 18 are views of a plate positioner and stabilizer (positioner/stabilizer) of the present disclosure, which can be used to position and stabilize the adjustable rib plate apparatus of FIG. 4A.

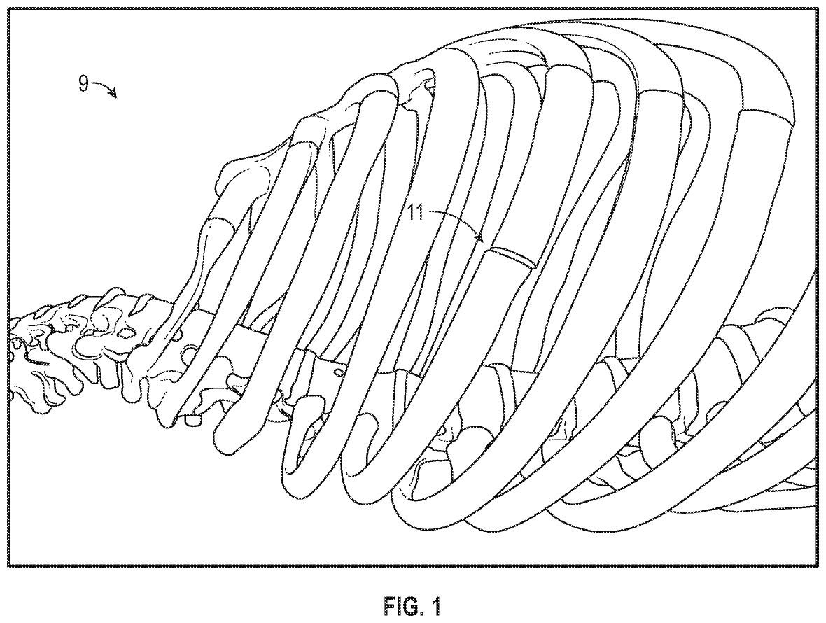

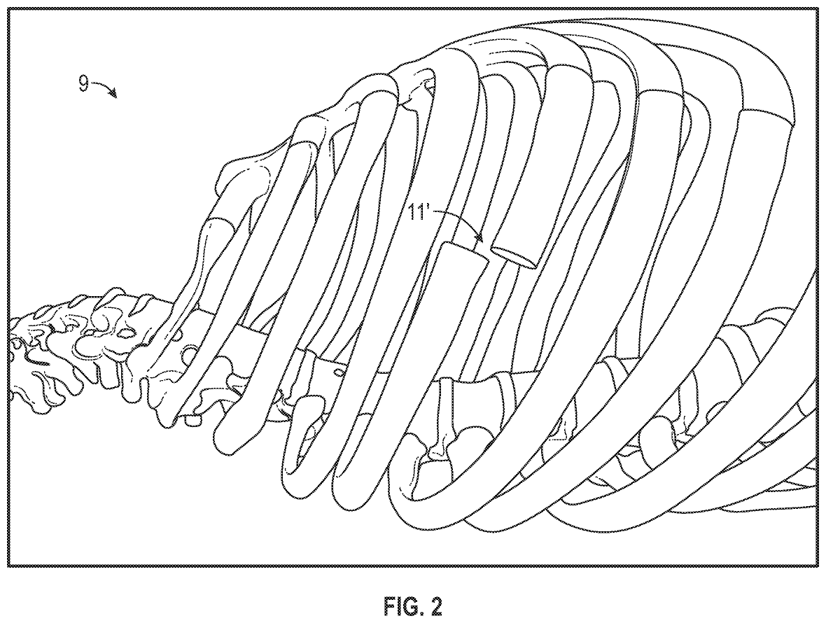

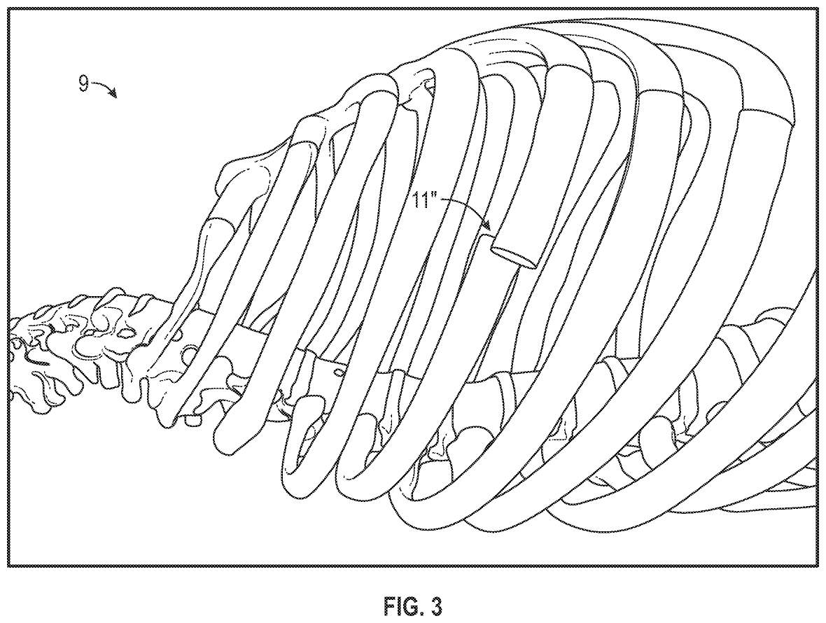

### DETAILED DESCRIPTION OF EMBODIMENTS

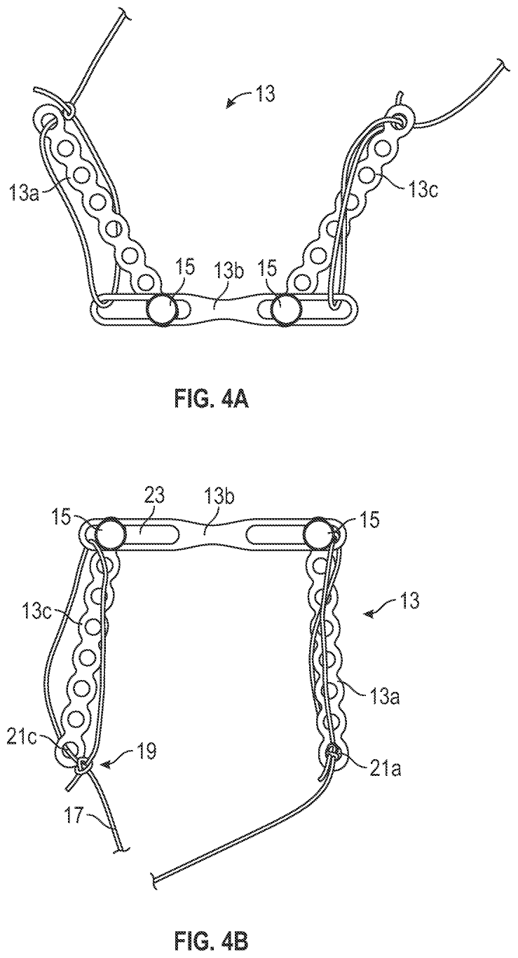

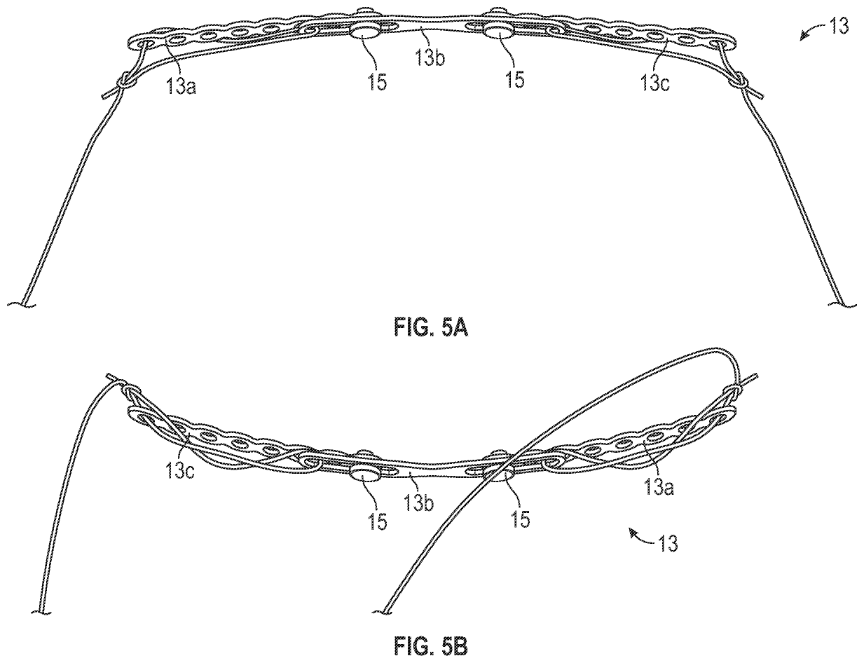

FIGS. 4 through 6 illustrate an adjustable rib plate apparatus 13 in accordance with one embodiment, among others, of the present disclosure. With a standard conventional rib plate, there is a linear plate of metal that can be bent to fit whatever configuration of the rib is needed. Longer plates generally provide greater stability than short plates. However, deep within a submuscular cavity, such as under the scapula or the large muscle groups within the back, placing a large plate becomes very difficult. In an effort to drive minimally invasive surgery forward and create less trauma to the already traumatized patient, the inventor has created the adjustable rib plate apparatus 13 of FIGS. 4 through 6. This adjustable rib plate apparatus 13 can be essentially collapsed into a more compact configuration and placed within a smaller, more confined area.

Standard rib fixation requires the use of multiple instruments and many hands of the surgeon and assistants, which overcrowds the limited operative field and exposure. Sometimes, counter incisions or very large incisions are required in order to fixate a rib appropriately. Placing a displaced rib fracture 11′ or telescoped rib fracture 11″ into a correct configuration for plating often is very cumbersome and difficult. The adjustable rib plate apparatus 13 of the present disclosure addresses those issues. It is a three-plate construct having connected but movable plates 13a, 13b, 13c. The adjustable rib plate apparatus 13 can be adjusted to an extended configuration or a more compressed, or collapsed, configuration. When extended and deployed fully, this provides a significant amount of stability as the three plates 13a, 13b, 13c become a single, linear construct, all within the same plane. The two outer plates 13a, 13c that rotate about a center plate 13b with sliding holes 23 utilize axles 15 to allow complete range of motion. Moreover, each of the outer plates 13a, 13c has a series of screw holes to enable fixation to a rib fragment.

Each axle 15 has cylindrical slidable portion that extends from a surface of a respective plate 13a, 13c and that permits sliding of the respective plate 13a, 13c along a respective elongated hole 23. Each axle 15 also includes a circular end stop that is larger in diameter than the cylindrical slidable portion and that secures the cylindrical slidable portion in the elongated hole 23.

The three plates 13a, 13b, 13c can be made of any suitable material, such as a metal, that makes them sufficiently rigid but bendable.

Referring to FIG. 4B, alignment cables 17 with respective slip knots 19 are placed through the respective outer holes 21a, 21c of each of the outer side plates 13a, 13c and through an elongated sliding hole 23 of the center plate 13b. Each alignment cable 17 with slip knot 19, as it is pulled and tightened by the surgeon, draws its respective outer plate 13a, 13c and the center plate 13b into alignment with one another. As they realign each other, they will right and realign the randomly configured, displaced rib segments to which they are temporarily affixed. After two screws are placed into each fractured rib fragment through each of the side plates 13a, 13c, the slip knots 19 can be pulled to draw the plates 13a, 13b, 13c into linear alignment, automatically bringing the displaced rib fragments into a normal alignment.

In addition to a minimally invasive external submuscular approach, a secondary goal of the adjustable rib plate apparatus 13 is to provide a plating system to be used from a completely internal approach, through video-assisted thoracoscopic surgery (VATS). Nearly the entire ribcage 9 is visible from inside of the chest on the inner surface. The fractures 11, 11′, 11″ can be easily and accurately located from that position, and there is essentially no muscle dissection that is required to access them from an internal approach; however, this current technology and idea are limited. There are no plating systems commercially available that address rib fractures completely internally.

With the video-assisted thoracoscopic approach, an incision can be placed in any portion of the ribcage 9 between two ribs and can be made to be about 3 cm long. With a standard wound protector, the small incision can be temporarily enlarged. This almost completely avoids muscle division in an already traumatized patient. Notably, the internal adjustable rib plate apparatus 13 is designed slightly differently than its external, submuscular counterpart. Both have the same configuration of a three-plate construct. The external adjustable rib plate apparatus 13 is arranged so that the center plate 13b and the end stops of the axles 15 are located outwardly and away from the convex rib surface. To properly contact the surface of a rib, the inside surface of the plate construct would need to be smooth.

With an internal approach, the opposite is performed. The internal adjustable rib plate apparatus 13 would position the center plate 13b and the end stops of the axles 15 inwardly and away from the concave internal surface of the rib. Both plate apparatus designs would be preconfigured and curved according to the curvature of the rib surface to which they are intended to be applied. With external plating, the adjustable rib plate apparatus 13 is bent to be convex. With internal plating, the adjustable rib plate apparatus 13 is bent to be concave.

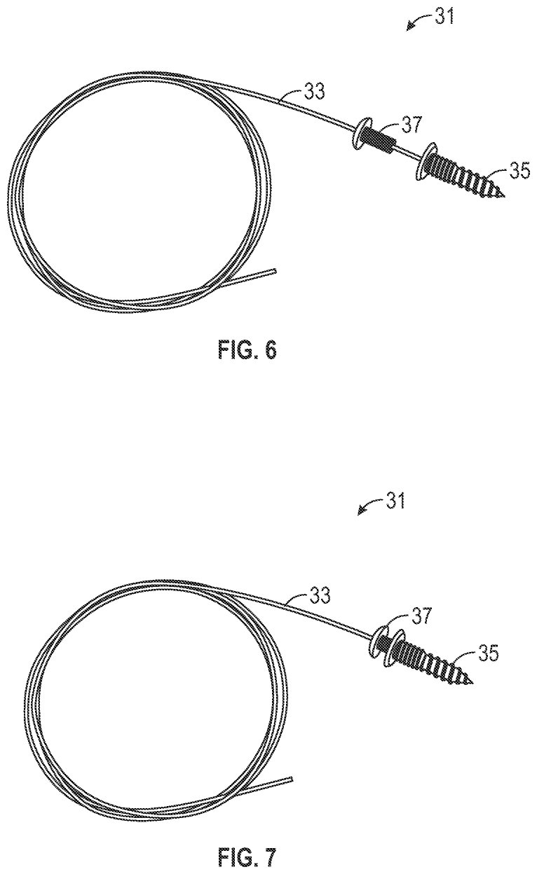

With either the submuscular approach or the internal thoracoscopic approach, the initial placement of the plate 13 and screws can be very difficult. Guiding the plate 13 into an exact, correct configuration from a remote location outside of the incision can be very cumbersome using current technology. To alleviate this issue, the inventor created a guidance screw assembly 31, one of which is illustrated in FIGS. 6 through 8. During a surgical procedure, the first screws to be installed are the guidance screws 35 associated with respective guidance screw assemblies 31. The guidance screws 35 are driven into the rib fragments at a premeasured distance from the fracture point. Guidance screws 35 are first installed in two of the outer screw holes in each of the outer plates 13a, 13c of the adjustable rib plate apparatus 13. A guidance cable 33 attached to the head of each guidance (or bone) screw 35 is placed through a screwdriver center cavity. In the preferred embodiment, the guidance cable 33 is wedged into the head. The screw 35 can then be driven into the rib while it is held in place like a leash so it is not lost in the depths of the surgical cavity. Additionally, the guidance cable 33 provides a convenient system that essentially leads the screwdriver 41 and the plate 13 directly to the prepositioned screw 35 where the surgeon intends for it to be placed without significant manipulation.

After the plate 13 is guided onto the guidance cable 33 and driven into place, a locking screw 37 tightens into an inner threaded cylindrical cannula of the head of the guidance screw 35. The cable 33 is then cut off or otherwise removed from the guidance screw 35. Preferably, the guidance cable 33 is made of a suitable material to make it capable of then being pulled off like a pop-off suture with a grasping instrument, and all that remains behind is the plate 13 and guidance screw 35 (with locking screw 37).

For initial placement of the adjustable rib plate apparatus 13 using the guidance screws 35, the first step is to measure the distances from the outermost and second outermost screw holes to the center of the plate construct axis, or center of the center plate 13b. Those distances are then either measured or approximated internally and the guidance screws 35 are then placed in the appropriate locations, with respect to the rib fracture point. Guidance screws 35 are then driven in place at the measured points along each fragment of the rib. The ends of the cables 33 that extend externally are then placed through the appropriate hole in the outer plates 13a, 13c. The plate construct is then guided along the multiple cables 33 into place in the surgical cavity to meet the previously placed guidance screws 35. After that, the locking screws 37 are then placed onto the guidance cables 33, and they fall essentially right into place against the surface of the plate 13 opposite the heads of respective guidance screws 35. A screwdriver, or cannulated screwdriver 41 in the preferred embodiment, is then placed sequentially over each of the cables 33 and used to lock in place the locking screws 37 against the guidance screws 35. Once this is complete, additional locking screws can be placed through the additional aligned holes of the outer plates 13a, 13b and sliding hole 23 of the center plate 13b after reduction and proper alignment of the rib has occurred by tightening the slip knots 19 by pulling the cables 17 (FIG. 4B).

It should be noted that the guidance screw assemblies 31 can be used in conjunction with the adjustable rib plate apparatus 13 of the present disclosure or with standard commercially available rib plates.

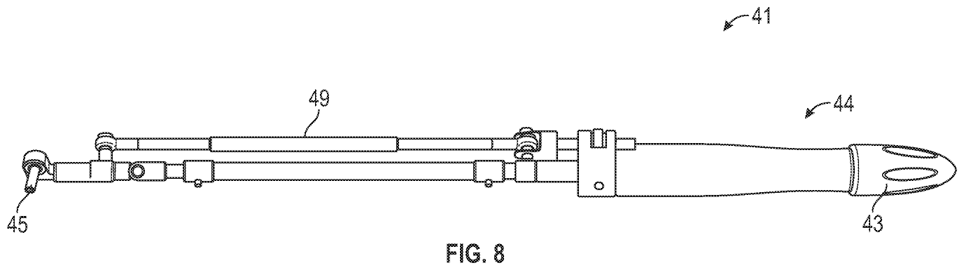

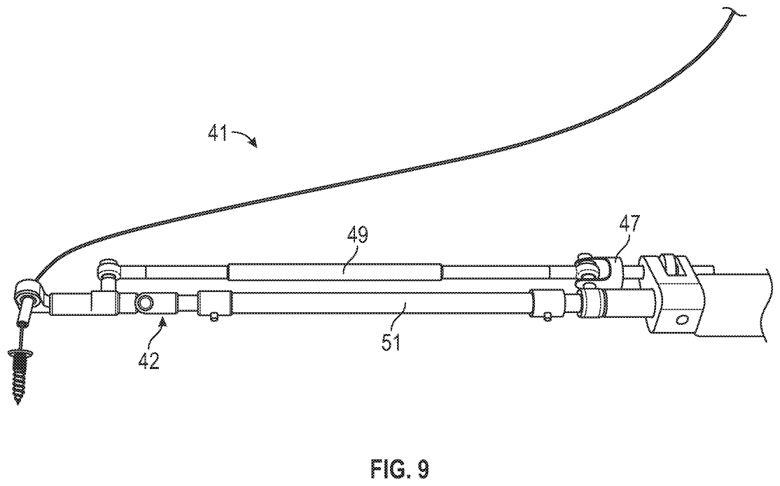

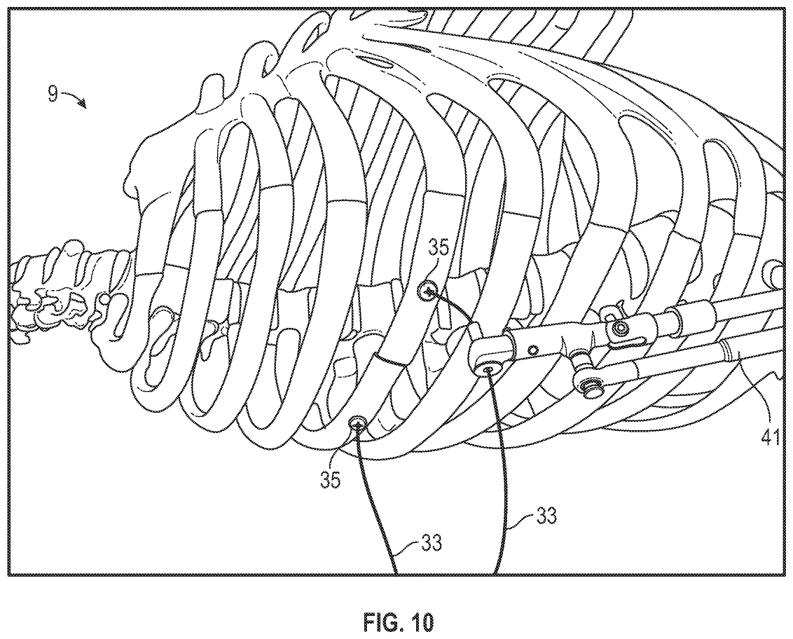

FIGS. 8 through 13 illustrate an extended steerable screwdriver 41 that can be used in connection with installing the adjustable rib plate apparatus 13 of the present disclosure. The steerable screwdriver 41 is a manual hand-operated driver, but can, in some embodiments, be a powered driver. It has an extended length on it to be able to fit deep within a submuscular wound, internally inside the thorax, etc. As a thumb screw 43 of the handle 44 is actuated and rotated remotely, the screwdriver 41 turns a ninety-degree offset, driver tip 45. This tip 45 is cannulated through the center to be able to accept the guidance cable 33 of the guidance screw 35. This screwdriver 41 is also steerable. Rarely is there only a straight or 90-degree pitch required deep within a submuscular or intrathoracic surgical cavity. To be able to drive screws 35 at random angles, this flexible driver tip 45 can be adjusted into various angles. It can be actuated externally at any angle desired.

The extended steerable screwdriver 41 has an extended shaft 51 with a worm gear 42 that drives the tip 45 at a ninety-degree angle. The worm gear 42 enables the movement of the tip 45. The tip 45 is also removable.

There is a universal joint 47 on a low-profile sway bar 49 parallel to the extended shaft 51 of the screwdriver 41. In an alternative embodiment, the sway bar 49 may be included within the housing of the extended shaft 51 of the screwdriver 41. When the thumb screw 43 is turned, depending upon the rotational direction, the sway bar 49 is either shortened or lengthened so that it engages and actuates the bend of the screwdriver 41, thereby allowing it to be guided and steered appropriately within the surgical cavity.

An inner drive shaft couples the driver's handle to the worm gear 42 and driver bit 45 at the opposite end of the screwdriver 41. The bend of the driver internally incorporates a segment of braided wire or other flexible material that is in linear contiguity with the rigid portions of the drive shaft. The flexible segment is sufficiently axially rigid to tolerate the strong rotational forces required to drive screws 35 into bone. The flexible segment is retained within a joint near the distal end of the instrument. The joint is constrained to freedom of motion only within a single plane to allow for up and down adjustment of the driver tip 45. Side to side movement of the instrument tip 45 is accomplished simply by rotating the axis of the entire driver through pronation or supination of the surgeon's hand.

It should be noted that the steerable screwdriver 41 can be used in conjunction with the adjustable rib plate apparatus 13 of the present disclosure or with standard commercially available rib plates and screws.

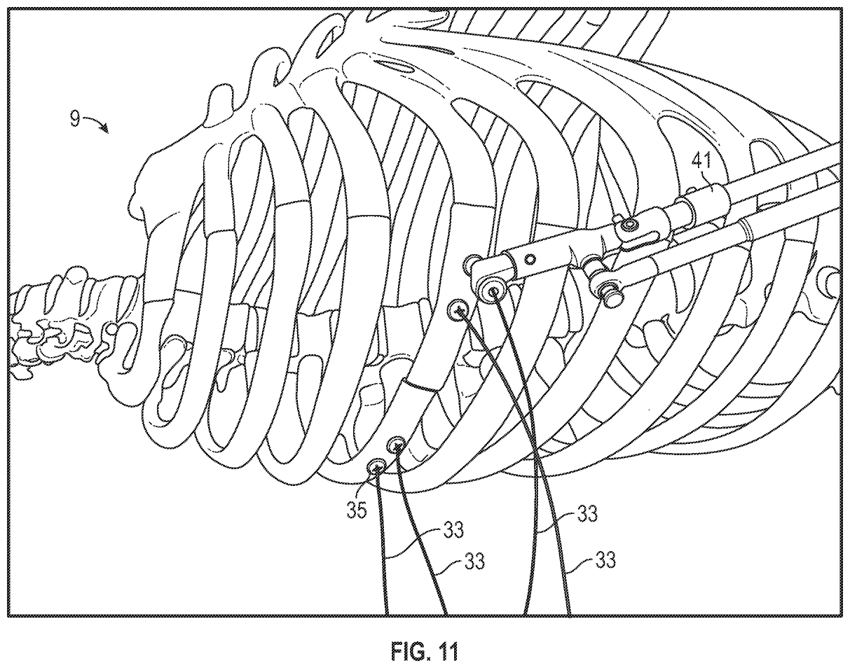

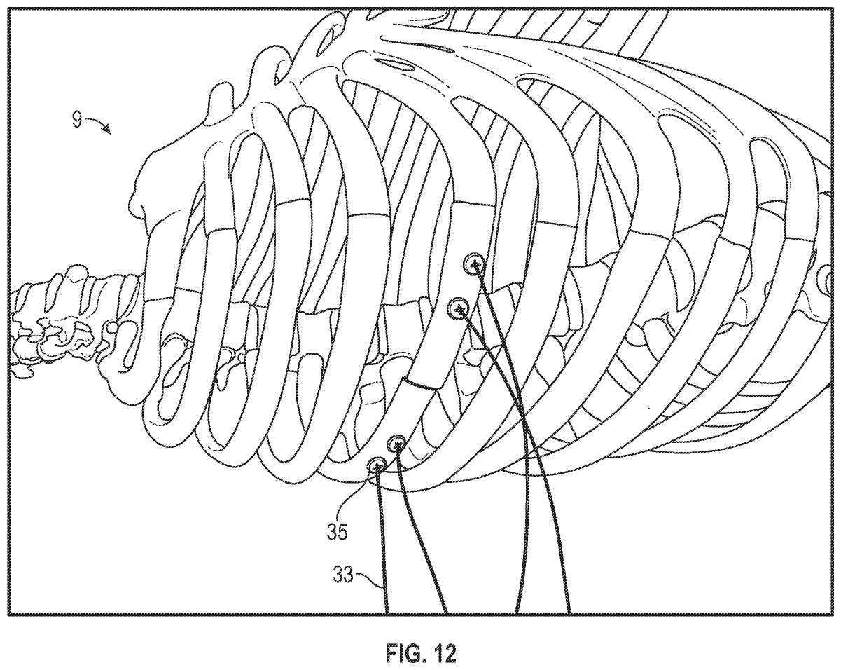

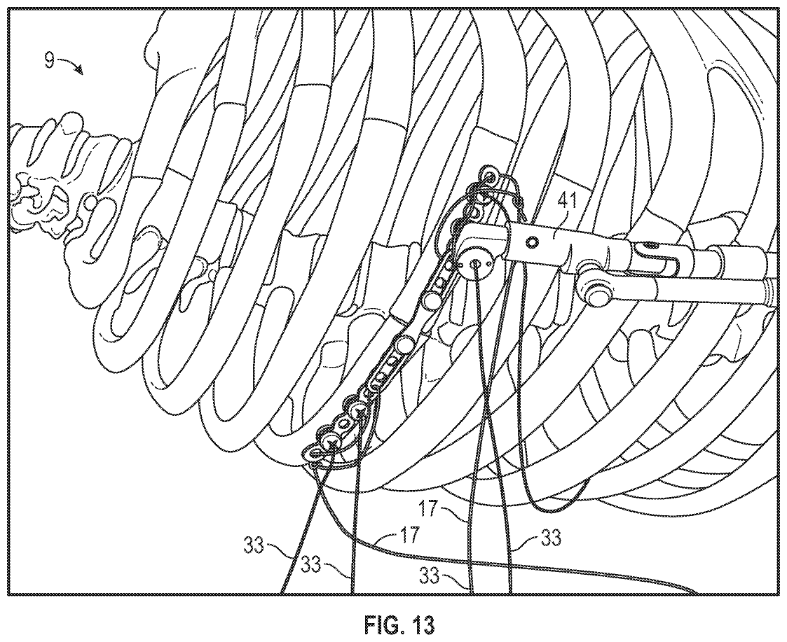

The overall method, or medical procedure, for repairing the fracture using the screwdriver 41 will now be described with reference to FIGS. 10 through 14. The guidance cable 33 of the guidance screw assembly 31 is initially placed through the center cannula of the driver tip 45. The driver tip 45 is engaged with and into the cruciform shape of a guidance screw 35. Note that the guidance cable 33 is also utilized as a tether to hold the screw 35 in place so that it is not lost within the cavity and is still free to turn. The guidance screw 35 is initially driven into a rib fragment at the premeasured and marked site in relation to the fracture point. This is ideally repeated to place two guidance screws 35 into the rib fragments on each side of the fracture, as shown in FIG. 11. The screwdriver 41 is then removed, as shown in FIG. 12.

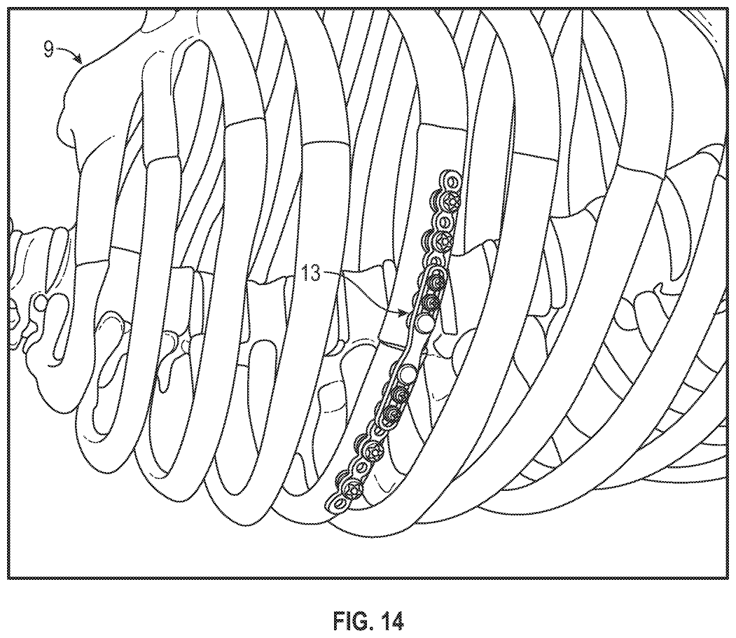

Next, the guidance cables 33 placed through the appropriate four plate holes in plates 13a, 13c of the apparatus 13, and the adjustable rib plate apparatus 13 is guided into the cavity using the guidance cables 33 and positioned appropriately against the heads of the guidance screws 35. The adjustable rib plate apparatus 13 will be in a random configuration at this point until the adjustable rib plate apparatus 13 is screwed down and fully deployed. The locking screw 37 of the guidance screw assembly 31 is then placed over the guidance cable 33 and guided to the respective plate 13a, 13c. The screwdriver 41 is then placed over the cable 33, and navigated directly onto the locking screw 37, as illustrated in FIG. 14. All the sequentially stacked components are held together along the tethering cable and tightened up to lock into a stable configuration. This process is repeated until two guidance screws 35 are completed on each outer plate 13a, 13c, and then the screwdriver 41 is removed. The guidance cables 33 can then be removed as pop-offs using a grasping instrument. This leaves the plate construct in a stable position affixed to both ends of the displaced, fractured rib.

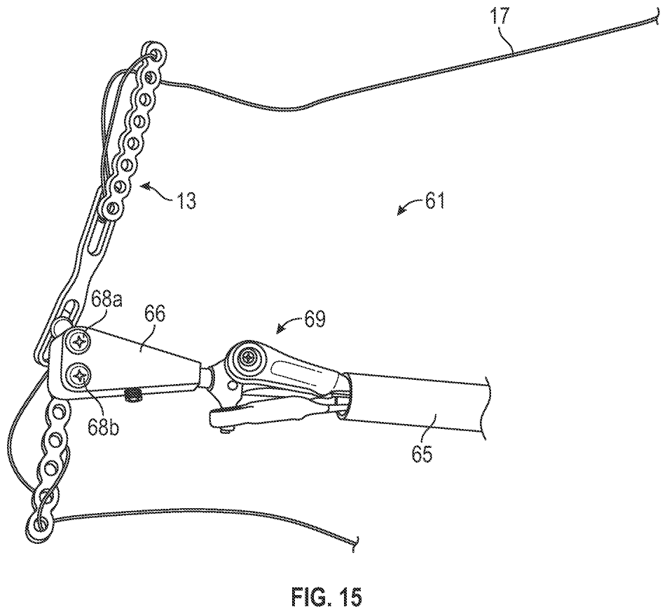

The alignment cables 17 with loops having slip knots 19 (FIG. 4B) are then pulled and tightened. As the loops are tightened, the plates 13a, 13b, 13c align into a single linear plane, which in turn reduces the displaced fracture and compresses the fragments together for final fixation. Finally, additional locking bone screws are placed through the aligned remaining holes in the three-plate construct. The center plate 13b is compressed against the outer plates 13a, 13c as the screws tighten them onto the rib fragments. The three plates 13a, 13b, 13c are thereby fused into one single plate structure that stabilizes the fracture. FIG. 15 shows the fully installed adjustable rib plate apparatus 13 on the rib fracture.

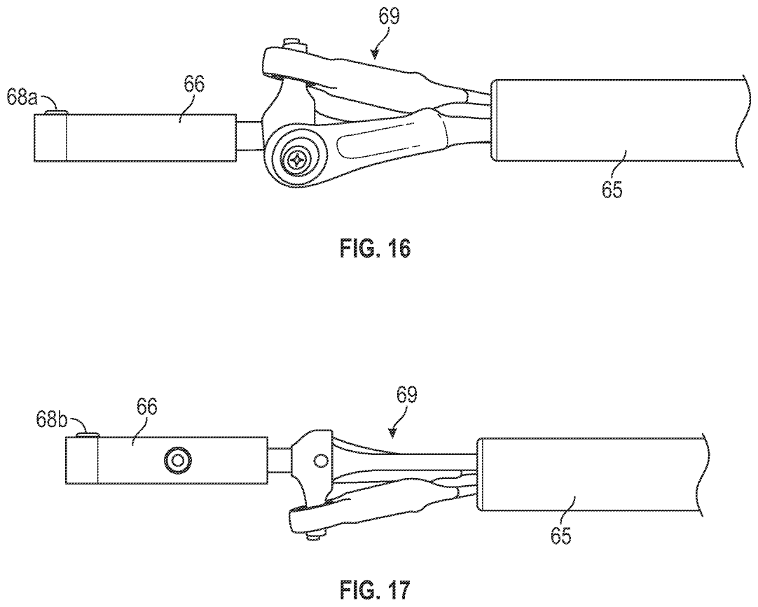

FIGS. 15 through 17 show a possible embodiment, among others, of a plate positioner and stabilizer (positioner/stabilizer) 61 of the present disclosure. The plate positioner/stabilizer 61 can be used when additional support or positioning assistance is needed. It is designed to detachably connect to and hold onto the adjustable rib plate apparatus 13 in order to assist with moving it and holding it in place while screws are placed through it. The plate positioner/stabilizer 61 has adjustable controls on the handle to enable external control of pitch, yaw, and roll movements of the adjustable rib plate apparatus 13.

The plate positioner/stabilizer 61 advances rib fracture repair by minimizing surgical trauma in patients already suffering from traumatic injuries. Current techniques usually require large, muscle dividing external incisions and do not allow rib fracture plating in hard-to-reach areas of the chest, like the area covered by the scapula and the heads of the ribs adjacent to the spine. The plate positioner/stabilizer replaces several instruments currently required for the same maneuvers in external muscle-sparing rib plating exposures. It also provides a means of positioning the plate 13 into proper orientation inside the chest when a thoracoscopic approach is used. Of note, thoracoscopic methods of rib fracture plating are currently in their infancy and no similar instrument is currently in use.

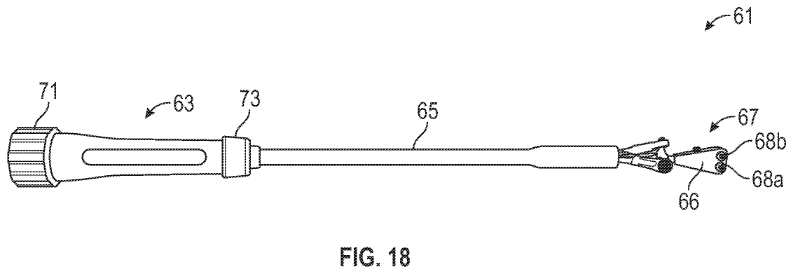

As shown in FIGS. 15 through 18, the plate positioner/stabilizer 61 has an elongated body extending between a proximate end and a working distal end. The proximate end includes an external handle 63. An elongated shaft 65 extends from the external handle 63 to its distal working end 67. Its distal working end 67 is designed to be low-profile for placement and maneuvering within a deep submuscular surgical exposure of fractured ribs, or totally intrathoracic via a small utility incision between the ribs. The distal working end 67 includes a positioner head 66 with two screws 68a, 68b that temporarily and removably affixes the adjustable rib plate apparatus 13 to a ball joint 69 on the instrument's extreme distal end.

The shaft 65 of the instrument 61 and handle 63 are designed to be in line. The handle 63, which is controlled from outside of the limited-access surgical cavity, has tandem twist knobs 71, 73 that each control one of two parallel shafts within the center channel of the instrument's shaft 65. Turning one knob 71, 73 results in pitch movement of the ball joint to which the plate is affixed. Turning the other knob 73, 71 results in yaw movement. Twisting the entire instrument 61 within the surgeon's hand results in roll movement. Therefore, the adjustable rib plate apparatus 13 can be controlled remotely in three dimensions to position the apparatus 13 perfectly across a rib fracture. After the plate is positioned appropriately, the instrument 61 is simply held in place to stabilize the rib plate 13 while bone screws are placed for fixation.

It should be noted that the plate positioner/stabilizer 61 can be used either in conjunction with the adjustable rib plate apparatus 13 of the present disclosure or with standard commercially available rib plates and screws.

While in the foregoing specification this invention has been described in relation to certain embodiments thereof, and many details have been put forth for the purpose of illustration, it will be apparent to those skilled in the art that the invention provides many additional embodiments and that certain of the details described herein can be varied considerably without departing from the basic principles of the invention.

At least the following is claimed:

1. An adjustable rib plate apparatus, comprising:
a center plate having an elongated body with opposing sides that are generally planar, the opposing sides having a periphery defined by opposing side edges and opposing ends;
a first outer plate, the first outer plate having an elongated body with opposing sides that are generally planar, the opposing sides having a periphery defined by opposing side edges and opposing ends;
a second outer plate, the second outer plate having an elongated body with opposing sides that are generally planar, the opposing sides having a periphery defined by opposing side edges and opposing ends;
a first attachment means for attaching the first outer plate to the center plate at a location near respective ends of the first outer plate and the center plate, the first attachment means for permitting the first outer plate to be movable in a geometric plane that is generally parallel to the geometric planes associated with the opposing sides of the center plate; and
a second attachment means for attaching the second outer plate to the center plate at a location near respective ends of the second outer plate and the center plate, the second attachment means for permitting the second outer plate to be movable in a geometric plane that is generally parallel to the geometric planes associated with the opposing sides of the center plate.

2. The apparatus of claim 1, wherein:
each of the first and second outer plates comprise a plurality of screw holes extending between the opposing sides; and
the center plate comprises elongated first and second sliding holes, each near a respective opposing end of the center plate and each having a linear channel extending parallel to the opposing side edges;
a first axle that secures the first outer plate in the first sliding hole and permits movement of the first outer plate relative to the center plate within the linear channel of the sliding hole and rotationally about the sliding hole; and
a second axle that secures the second outer plate in the second sliding hole and permits movement of the second outer plate relative to the center plate within the linear channel of the sliding hole and rotationally about the sliding hole.

3. The apparatus of claim 2, further comprising first and second alignment cable loops with slip knots attached to the adjustable rib plate apparatus, the first alignment cable loop passing through the first sliding hole of the center plate and an outmost screw hole of the first outer plate, the second alignment cable loop passing through the first sliding hole of the center plate and an outmost screw hole of the second outer plate.

4. The apparatus of claim 1, further comprising a fractured rib having first and second parts and wherein the first and second outer plates are screwed to the first and second parts, respectively, so that a first side of the outer plates is contiguous with one of the parts and a second side of the outer plates is contiguous with the center plate.

5. The apparatus of claim 1, wherein the first outer plate, second outer plate, and centerplate are bendable.

6. The apparatus of claim 1, further comprising a guidance screw assembly, the guidance screw assembly comprising:
a first screw, the first screw having an elongated body with turning head and tip at opposing ends, the body having outer threads, the first screw having an inner cannula with inner threads;
a second screw, the second screw having an elongated body with a turning head and tip at opposing ends, the body having outer threads that match the inner threads so the second screw can be driven into the cannula;
a segment of flexible cable extending through the elongated body of the second screw between the turning head and the tip, the cable further extending to and detachably secured to the first screw; and
wherein the tip of the first screw extends into part of a rib.

7. A method for repairing a rib fracture, comprising the steps of:
(a) providing an adjustable rib plate apparatus as defined in claim 2;
(b) providing first and second pairs of guidance screw assemblies, each guidance screw assembly comprising:
(1) a bone screw, the bone screw having an elongated body with turning head and tip at opposing ends, the body having outer threads, the bone screw having an inner cannula with inner threads;
(2) a locking screw, the locking screw having an elongated body with a turning head and tip at opposing ends, the body having outer threads that match the inner threads so the locking screw can be driven into the cannula; and
(3) a segment of flexible guidance cable extending through the elongated body of the locking screw between the turning head and the tip, the cable further extending to and detachably secured to the bone screw;
(c) attaching first and second alignment cables having alignment cable loops with slip knots to the adjustable rib plate apparatus, the first alignment cable loop passing through the first sliding hole of the center plate and an outmost screw hole of the first outer plate, the second cable loop passing through the first sliding hole of the center plate and an outmost screw hole of the second outer plate;
(d) installing each of the bone screws of the first and second pairs of guidance screws into first and second rib parts, respectively;
(e) passing each of the guidance cables from the first and second pairs of guidance screws, respectively, through first and second screw holes of the first and second outer plates, respectively, and guiding the adjustable rib apparatus against the first and second rib parts;
(f) guiding each of the locking screws with respective guidance cables to respective bone screws and securing the first and second outer plates of the adjustable rib plate apparatus to the first and second rib parts, respectively, by installing the locking screws into the bone screws;
(g) detaching the guidance cables from the bone screw and the locking screw and removing the guidance cables;
(h) tightening the slip knot alignment cable loops in order to bring the first and second rib parts in closer alignment by pulling on the alignment cables;
(i) installing a plurality of additional bone screws through respective screw holes in the first and second outer plates and into the first and second rib parts, respectively; and
(j) detaching the alignment cables from the adjustable rib plate apparatus and removing the alignment cables.

8. The method of claim 7, further comprising the step of using an extended steerable screwdriver to install the bone screws and the locking screws, the extended steerable screwdriver comprising:
an elongated body extending between first and second ends;
a rotatable handle situated at the first end of the elongated body;
a drive shaft that passes through the elongated body and is rotated by the handle;
a flexible segment associated with the drive shaft, the flexible segment enabling the drive shaft to bend;
a worm gear that is driven by the drive shaft;
an offset driver having a removable cannulated tip, the worm gear driving the offset driver, the tip designed to matingly engage and drive a turning head of a guidance screw while enabling a tether cable associated with the guidance screw to extend from the turning head of the guidance screw into, through, and out of the driving tip; and
an elongated sway bar extending along the elongated body, the sway bar having a universal joint with thumb screw knob designed so that when the thumb screw knob is turned, the sway bar is inwardly or outwardly moved so that the sway bar angularly bends the driver at the flexible segment so that the driver can be guided and steered angularly.

9. The method of claim 7, further comprising the step of using a plate positioner and stabilizer to guide the adjustable rib apparatus against the first and second rib parts; the plate positioner and stabilizer comprising:
an elongated body extending between a proximate end and a working distal end;
first and second elongated shafts extending along the body;
a handle with first and second control mechanisms, the first and second control mechanisms designed to respectively and independently move the first and second shafts in a longitudinal direction along the body; and
a positioner head that is attached to and that is pivoted by the first and second elongated shafts when moved in the longitudinal direction, the positioner head having a plurality of screws, the screws for detachably mounting to the rib plate.

10. The method of claim 9, wherein the positioner head further comprises a universal joint that is controlled by the first and second elongated shafts and that enables the positioner head to be moved in respectively a pitch direction and a yaw direction, and wherein movement in a roll direction is accomplished by a surgeon when the handle is twisted.

11. The method of claim 10, wherein the control mechanisms are each a twist knob on the handle, each being rotational about an axis that runs along the longitudinal body.

12. An adjustable rib plate apparatus, comprising:
a center plate having an elongated body with opposing sides that are generally planar, the opposing sides having a periphery defined by opposing side edges and opposing end, the center plate comprising elongated first and second sliding holes, each near a respective opposing end of the center plate and each having a linear channel extending parallel to the opposing side edges;
a first outer plate, the first outer plate having an elongated body with opposing sides that are generally planar, the opposing sides having a periphery defined by opposing side edges and opposing ends, the first outer plate comprising a plurality of screw holes extending between the opposing sides;
a second outer plate, the second outer plate having an elongated body with opposing sides that are generally planar, the opposing sides having a periphery defined by opposing side edges and opposing ends, the second outer plate comprising a plurality of screw holes extending between the opposing sides;
a first axle that secures the first outer plate in the first sliding hole and permits movement of the first outer plate relative to the center plate within the linear channel of the sliding hole and rotationally about the sliding hole; and
a second axle that secures the second outer plate in the second sliding hole and permits movement of the second outer plate relative to the center plate within the linear channel of the sliding hole and rotationally about the sliding hole.

13. The apparatus of claim 12, further comprising first and second alignment cable loops with slip knots attached to the adjustable rib plate apparatus, the first alignment cable loop passing through the first sliding hole of the center plate and an outmost screw hole of the first outer plate, the second alignment cable loop passing through the first sliding hole of the center plate and an outmost screw hole of the second outer plate.

14. The apparatus of claim 12, further comprising a fractured rib having first and second parts and wherein the first and second outer plates are screwed to the first and second parts, respectively, so that a first side of the outer plates is contiguous with one of the parts and a second side of the outer plates is contiguous with the center plate.

15. The apparatus of claim 12, wherein the first outer plate, second outer plate, and centerplate are each bendable.

16. The apparatus of claim 12, further comprising:
a first screw, the first screw having an elongated body with turning head and tip at opposing ends, the body having outer threads, the first screw having an inner cannula with inner threads;
a second screw, the second screw having an elongated body with a turning head and tip at opposing ends, the body having outer threads that match the inner threads, the second screw being threaded in the cannula; and
wherein the tip of the first screw extends into part of a rib.

17. A method for rib fracture fixation, comprising the steps of:
providing an adjustable rib plate apparatus with an elongated center plate and first and second elongated outer plates, the outer plates having a body extending between first and second opposing ends, the first ends being attached to but movable relative to the center plate in a two dimensional plane associated with the center plate;
installing a pair of bone screws with detachable guidance cables on each of two bone fragments on separate sides of a fracture;
installing removable first and second alignment cable loops with slip knots on the adjustable rib plate apparatus by extending the first loop around a part of the first outer plate and the center plate and the second loop around a part of the second outer plate and the center plate;
guiding the outer plates with respective guidance cables to respective destinations on the bone fragments;
guiding locking screws using respective guidance cables to respective heads of respective bone screws and securing the locking screws to the heads while also securing the outer plates to the bone fragments;
aligning the center plate with the outer plates as well as aligning the bone fragments of the fracture by pulling the on the first and second alignment cable loops; and
installing additional bone screws in the first and second outer plates to fully fixate the fracture.
